(19) United States
(12) Patent Application Publication
(10) Pub. No.: US 2026/0071983 A1
(43) Pub. Date: Mar. 12, 2026

(54) METHOD TO IDENTIFY AND QUANTIFY TIRE WEAR MICROPLASTICS

(71) Applicants: Kanchana Chandrakanthan, Tempe, AZ (US); Pierre Herckes, Gilbert, AZ (US); Matthew Fraser, Phoenix, AZ (US)

(72) Inventors: Kanchana Chandrakanthan, Tempe, AZ (US); Pierre Herckes, Gilbert, AZ (US); Matthew Fraser, Phoenix, AZ (US)

(21) Appl. No.: 19/323,555

(22) Filed: Sep. 9, 2025

Related U.S. Application Data
(60) Provisional application No. 63/692,501, filed on Sep. 9, 2024.

(51) Int. Cl. G01N 25/00; G01N 1/44; G01N 30/68; G01N 33/44

(52) U.S. Cl. CPC G01N 25/00; G01N 1/44; G01N 30/68; G01N 33/442

(57) ABSTRACT

Identifying and quantifying tire wear includes heating a sample including particulate matter obtained from tire wear and soot to a first temperature to thermally desorb an amount of organic carbon from the sample, cooling the sample from the first temperature to a second temperature, heating the sample from the second temperature to a third temperature to thermally desorb an amount of elemental carbon from the sample, and identifying an amount of tire wear particulate matter in the sample based on the amount of elemental carbon in the sample.

### CROSS-REFERENCE TO RELATED APPLICATION

This application claims the benefit of U.S. Patent Application No. 63/692,501 filed on Sep. 9, 2024, which is incorporated by reference herein in its entirety.

### TECHNICAL FIELD

This invention relates to a method of quantifying tire wear particles in aerosol samples.

### BACKGROUND

Tire wear microplastics form through mechanical abrasion between vehicle tires and road surfaces. It is estimated that tire wear particles may contribute up to about 60% of microplastic emissions. An accurate assessment of tire wear particle levels in aerosol samples is confounded by the presence of soot particles, which are difficult to distinguish from tire wear particles.

### SUMMARY

The present disclosure provides a thermo-analytical method to identify and quantify tire wear microplastics and distinguish them from soot in the environment. The method relies on an approach in separating soot and tire wear particles based on where elemental carbon evolves in thermograms from programmed thermal analysis (PTA). The thermo-analytical method described herein identifies tire wear particles, and distinguishes them from soot particles in aerosol samples using PTA combined with an organic carbon-elemental carbon analysis. This method can distinguish soot samples obtained from different sources (e.g., trucks, lamp and diesel exhaust) relative to tire wear particles.

In a first general aspect, identifying and quantifying tire wear includes heating a sample including particulate matter obtained from tire wear and soot to a first temperature to thermally desorb an amount of organic carbon from the sample, cooling the sample from the first temperature to a second temperature, heating the sample from the second temperature to a third temperature to thermally desorb an amount of elemental carbon from the sample, and identifying an amount of tire wear particulate matter in the sample based on the amount of elemental carbon in the sample.

Implementations of the first general aspect can include one or more of the following features. In some cases, the first temperature is in a range of about 600° C. to about 900° C. Heating the sample to the first temperature can occur in the absence of oxygen. In some examples, heating the sample to the first temperature includes ramping a temperature of the sample from an initial temperature to the first temperature. In some implementations, the second temperature is in a range of about 500° C. to about 600° C. In some examples, the third temperature is in a range of about 600° C. to about 900° C. Heating the sample to the third temperature can occur in the presence of oxygen.

The first general aspect can further include oxidizing the organic carbon to yield carbon dioxide. In some cases, the first general aspect further includes converting the carbon dioxide to methane. In some implementations, the first general aspect further includes assessing an amount of the methane. Assessing the amount of the methane can include measuring the amount of methane using flame ionization detection. In some cases, assessing the amount of the methane includes integrating an area under a flame ionization detection curve between about 550° C. and about 630° C.

The first general aspect can further include oxidizing the elemental carbon to yield carbon dioxide. In some cases, the first general aspect further include converting the carbon dioxide to methane. In some implementations, assessing the amount of the methane includes measuring the amount of methane using flame ionization detection. Assessing the amount of the methane includes integrating an area under a flame ionization detection curve between about 630° C. and about 850° C. In some implementations, the first general aspect further includes adjusting the amount of methane to compensate for charring of the organic carbon in the sample. The first general aspect can include programmed thermal analysis using an organic carbon-elemental carbon analyzer.

### BRIEF DESCRIPTION OF DRAWINGS

FIG. 1 is a flow chart showing operations in a process for identifying and quantifying tire wear.

FIG. 2 shows an example PTA thermogram depicting the distinction between organic carbon (OC) and elemental carbon (EC).

FIG. 3 shows programmed thermal analysis (PTA) thermograms of tire wear and soot samples.

FIGS. 4A, 4B, and 4C show PTA thermograms of soot, tire wear particles, and carbon black particles, respectively.

FIG. 5 is a digital microscopic image of particles of the cryo-milled tire wear.

FIGS. 6A and 6B show PTA thermograms assessing the repeatability of tailpipe soot and tire wear samples, respectively.

FIG. 7 is a digital microscopic image of particles of PM₁₀ tire wear samples from the resuspension setup.

FIG. 8 shows PTA thermograms of PM_{2.5} samples from the Tyler St. parking structure, Tempe, AZ.

FIG. 9 shows PTA thermograms of aerosol samples collected from two tunnels (Jânio Quadros (JQ) tunnel and the tunnel no. 3 of the Beltway Rodoanel Mário Covas (RA)) in the São Paulo Metropolitan Area, Brazil.

FIG. 10 shows overlapped PTA thermograms of RA and JQ tunnel (modified temperature program) from 2011 samples.

FIG. 11 shows overlapped PTA thermograms of RA and JQ tunnel (modified temperature program) from 2018 samples.

### DETAILED DESCRIPTION

This disclosure describes an analytical method for identifying and quantifying tire wear particles in aerosol samples. The method includes separating soot and tire wear particles based on where elemental carbon evolves in thermograms from programmed thermal analysis (PTA).

Tire wear particles are often encrusted with mineral matter and bitumen, which increases the overall density of the particles. Separation techniques based on density may not account for this heterogeneity. Additional challenges arise when characterizing tire wear using spectroscopic methods. Tire wear particles typically include copolymers with additives including carbon black, which is a reinforcing filler used in tire manufacturing. Carbon black enhances the mechanical properties of the rubber polymers in tires, improving durability and strength. However, carbon black can cause strong fluorescence under laser light, thereby hindering chemical identification using spectroscopic methods such as micro-Raman spectroscopy. Thermo-analytical methods provide an alternative approach to analyzing and identifying tire wear microplastics.

Greater than 97% of carbon blacks constitute elemental carbon arranged as graphite layers to form primary particles. Soot is a similar refractory material, which is a carbonaceous substance produced during the incomplete combustion of hydrocarbons. Soot is composed primarily of elemental carbon and is often coated or embedded with other materials such as organic matter and sulfates to form internally mixed particles. The heterogenous reactivities of combustion products such as soot with other gaseous pollutants in the atmosphere, for example NO₂, can have implications in the atmosphere.

Refractory carbon or soot is typically analyzed in the atmosphere based on thermal optical transmittance and thermal optical reflectance. PTA allows for the differentiation of elemental carbon from organic carbon based on the thermal optical transmittance in the organic carbon-elemental carbon analyzer. Aerosol samples containing particulate matter on pre-fired quartz fiber filters are placed in the organic carbon-elemental carbon analyzer, and the sample is subjected to controlled heating cycles under selected temperature conditions. The method makes use of the thermal refractivity of elemental carbon, which does not volatilize at temperatures of approximately 700° C. in an inert atmosphere.

This disclosure describes a thermo-analytical method to distinguish between soot and tire wear in aerosol samples based on thermal optical transmittance using the organic carbon-elemental carbon analyzer. In an example, tire wear samples (n=8) are cryo-milled and thermally analyzed. Commercial grade carbon black used in tires and recovered carbon black from waste tires were used. Soot samples from different sources (e.g., trucks, lamp and diesel exhaust) were used to compare and contrast with tire wear samples. Repeatability measurements for tire wear and soot samples were conducted to assess the consistency of data, thereby ensuring the reliability of the measurements made on the organic carbon-elemental carbon analyzer. The effect of the size of the tire wear particle on the position where the high temperature thermal residue of elemental carbon appears in the thermogram was tested using PM₁₀ (particulate matter ≤10 μm) samples of tire wear. Aerosol samples collected from roads and urban areas typically contain a mixture of soot and tire wear particles. The method was applied on aerosol samples collected at a parking structure in Tempe, AZ and inside two tunnels located in the São Paulo Metropolitan Area, Brazil.

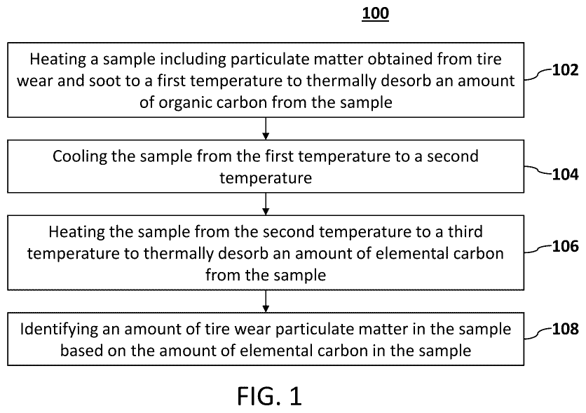

FIG. 1 is a flow chart showing operations in process 100 for identifying and quantifying tire wear. In 102, a sample including particulate matter obtained from tire wear and soot is heated to a first temperature to thermally desorb an amount of organic carbon from the sample. The first temperature typically is in a range of about 600° C. to about 900° C. In some cases, heating the sample to the first temperature occurs in the absence of oxygen. Heating the sample to the first temperature can include ramping a temperature of the sample from an initial temperature to the first temperature. In 104, the sample is cooled from the first temperature to a second temperature. The second temperature is typically in a range of about 500° C. to about 600° C. In 106, the sample is heated from the second temperature to a third temperature to thermally desorb an amount of elemental carbon from the sample. The third temperature is typically in a range of about 600° C. to about 900° C. In some implementations, heating the sample to the third temperature occurs in the presence of oxygen. In 108, an amount of tire wear particulate matter in the sample is identified based on the amount of elemental carbon in the sample.

In some cases, process 100 further includes oxidizing the organic carbon to yield carbon dioxide. Process 100 can further include converting the carbon dioxide to methane. In some implementations, process 100 further includes assessing an amount of the methane. In some examples, assessing the amount of the methane includes measuring the amount of methane using flame ionization detection. Assessing the amount of the methane can include integrating an area under a flame ionization detection curve between about 550° C. and about 630° C.

In some cases, process 100 further includes oxidizing the elemental carbon to yield carbon dioxide. Process 100 can further include converting the carbon dioxide to methane. In some implementations, process 100 further includes assessing an amount of the methane. Assessing the amount of the methane can include measuring the amount of methane using flame ionization detection. In some cases, assessing the amount of the methane includes integrating an area under a flame ionization detection curve between about 630° C. and about 850° C. Process 100 can further include adjusting the amount of methane to compensate for charring of the organic carbon in the sample. In some cases, process 100 includes programmed thermal analysis using an organic carbon-elemental carbon analyzer.

### EXAMPLES

### Tire Wear Sample Preparation

As shown in Table 1, tire wear samples (n=8) (CRM of America LLC, Mesa, AZ) were cut into smaller pieces of approximately 3 mm or smaller in diameter using clean, contamination free pliers. The cut tire wear samples were cryo-milled using a 6775 Cryogenic Grinder (Spex Sample Prep, NJ, USA) to produce micro-sized particles. The samples were transferred to small polycarbonate center cylinder vials with steel end plugs. The cryogenic grinding time was counted by cycles, where one cycle included 10 minutes of pre-cool time, 2 minutes of grinding at a rate of 10 counts per second (cps) and 2 minutes of rest.

The cryo-milled tire wear samples were transferred to pre-fired (650° C., overnight) quartz fiber filters (Whatman QM-A, Sigma Aldrich, MO, USA). Particle sizes of the cryo-milled tire wear were confirmed by examining under a digital microscope (Leica DM6B-Z, Germany) equipped with the Leica DFC7000 T camera and the Leica Application Suite X (LAS X) software. Soot samples from the tail pipes of trucks (n=4) and soot particles formed during the incomplete combustion of candle wax were similarly transferred to pre-fired quartz fiber filters for subsequent thermal analysis.

TABLE 1
Summary of samples analyzed

| Tire wear samples | Soot samples |
|---|---|
| Yokohama | Tailpipe (Truck 1) |
| Goodyear | Tailpipe (Truck 2) |
| Toyo | Tailpipe (Truck 3) |
| Bridgestone | Tailpipe (Truck 4) |
| Crumb rubber | Lamp |
| Hankook | Diesel exhaust |
| Michelin | |
| Nitto | |

Tire wear and soot samples were tested for consistency by taking measurements for repeatability of the same sample on multiple days. A soot sample from the tail pipe of a truck and a cryo-milled tire wear sample (Yokohama) were used for repeatability tests. Subsequent thermal analyses were performed.

A tire wear sample collected as debris from a highway road in Phoenix, AZ was cryo-milled using a 6775 Cryogenic Grinder (Spex SamplePrep, NJ, USA) with larger polycarbonate center cylinder vials and longer grinding times with increased number of cycles and cooling time to produce smaller tire wear particles. The cryo-milled tire wear sample was resuspended to obtain PM₁₀ size fractionated aerosols for subsequent programmed thermal analysis (PTA). The tire wear samples were placed in a clean Erlenmeyer flask and HEPA-filtered air was passed over the sample to resuspend relatively smaller particles. The resuspended particles were passed through a size-selective cyclone (URG Corporation, NC, USA) and collected on a downstream pre-fired quartz fiber filter (Whatman QM-A, Sigma Aldrich, MO, USA). The operating flow rate was 15.9 L min⁻¹ for the PM₁₀ sampling. The particle sizes of the collected PM₁₀ filter sample were confirmed by examining under a digital microscope (Leica DM6B-Z, Germany) equipped with the Leica DFC7000 T camera and the Leica Application Suite X (LAS X) software. The sample was subsequently analyzed using the organic carbon-elemental carbon analyzer.

A carbon black tire grade sample (N300) (Milagro Rubber Company, Inc., TX, USA) with a particle size range of 30 nm to 35 nm was transferred to a pre-fired quartz fiber filter and tested using the organic carbon-elemental carbon analyzer. Similarly, recovered carbon black samples (n=2) from waste tires (Contec, Warsaw, Poland) with particle diameters, d<12 μm and d<26 μm, were tested using the organic carbon-elemental carbon analyzer. The samples were analyzed using the organic carbon-elemental carbon analyzer as described.

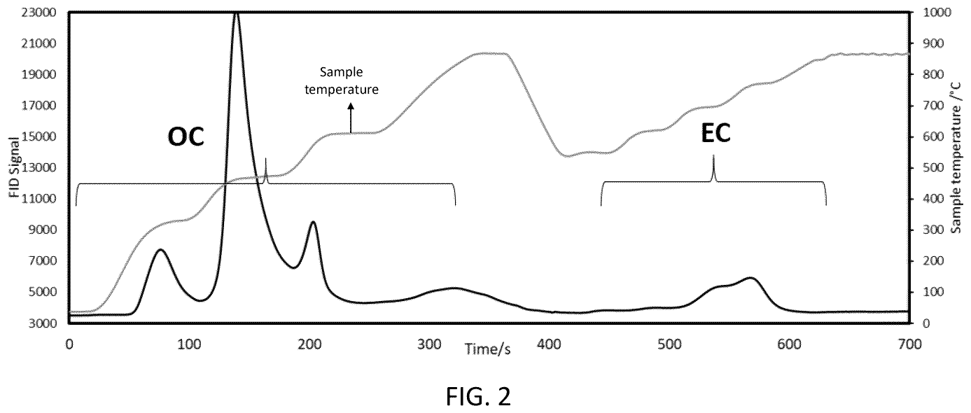

PTA was performed using an organic carbon-elemental carbon analyzer (Sunset Laboratory, Inc., Forest Grove, OR, USA). The heating process involving the different temperature ramps allowed the volatilization and oxidization of carbonaceous compounds in the sample, and organic carbon and elemental carbon were differentiated based on their distinct thermal characteristics. The sample was placed on a (1.5×1) cm punch of the quartz fiber filter and placed in the quartz oven. The oven was purged with helium (He) and a stepped temperature ramp increased the temperature in the quartz oven that led to the thermal desorption of organic compounds. The evolved carbon goes through an oxidizing oven and was quantitatively converted to carbon dioxide (CO₂). The carbon dioxide was swept out of the oven by He gas and mixed with hydrogen. The gaseous mixture was passed over a nickel catalyst to produce methane (CH₄). The CH₄ produced was measured using a flame ionization detector. The oven was cooled to a temperature of 550° C. after the initial temperature ramp and a second temperature ramp in the oxidizing He/O₂ carrier gas mixture was used to oxidize any elemental carbon present in the filter. Referring to FIG. 2, elemental carbon was determined in a manner similar to that used for the organic carbon. A red diode laser corrected the evolved elemental carbon measured. This was done by distinguishing the portion of elemental carbon that is naturally occurring in the sample from the portion that was formed by the charring of the organic carbon in the initial temperature ramp.

The samples on filters were analyzed with variable time steps lasting between 80 seconds and 110 seconds during the evolution of organic carbon at temperature plateaus of 310° C., 475° C., 615° C., and 870° C. The temperature profile during the evolution of elemental carbon included 45 second time step plateaus at 550° C., 625° C., 700° C., 775° C., and 850° C., with a final hold at 870° C. for 110 seconds.

### Example 1. Applying the Method on Urban Aerosol Samples

The method to distinguish soot and tire wear was applied on urban aerosol samples collected inside two road tunnels in the São Paulo Metropolitan Area.

Ambient aerosol PM_{2.5} samples were collected from the Tyler Street parking garage at the Tempe campus of Arizona State University, Tempe, AZ. The PM_{2.5} samples were collected using a high-volume air sampler (Tisch Environmental Inc., OH, USA) equipped with a PM_{2.5} impactor stage (TE 231, Tisch Environmental Inc., OH, USA) by drawing air through pre-fired (650° C., overnight) quartz fiber filters (Whatman QM-A, Sigma Aldrich, MO, USA) at a rate of 1.13 m³ min⁻¹.

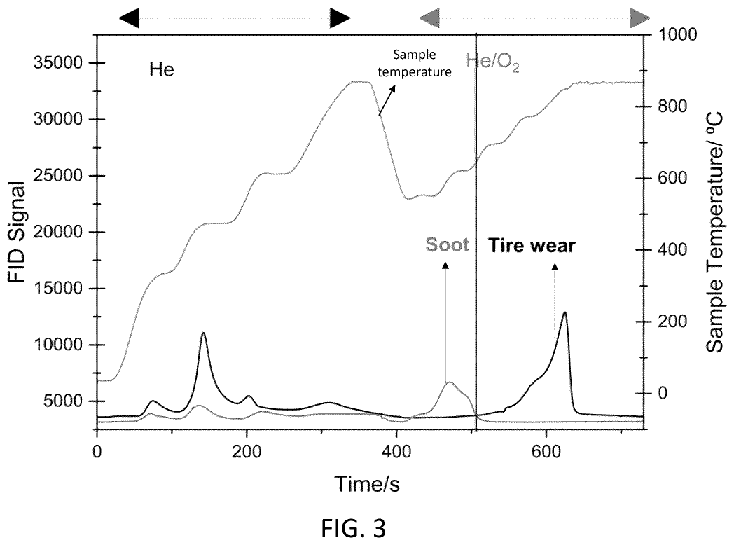

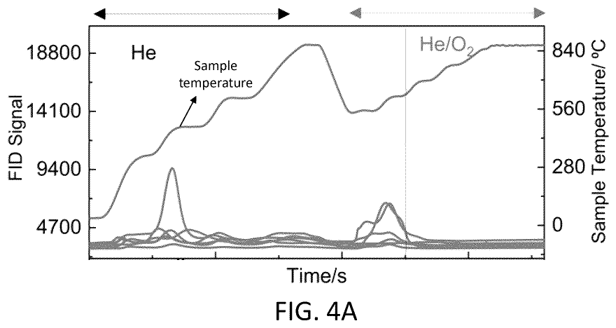

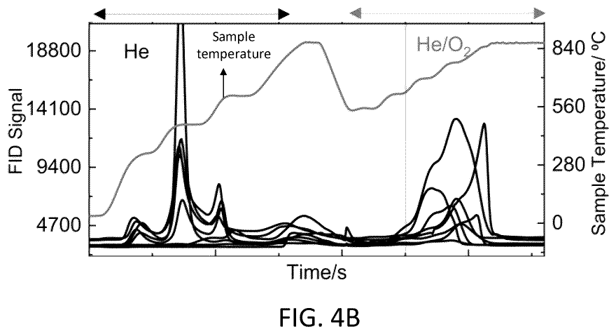

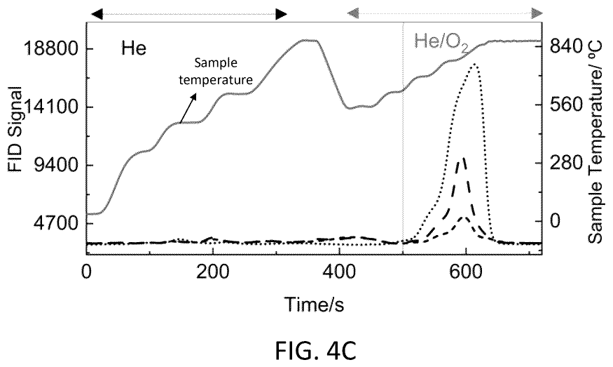

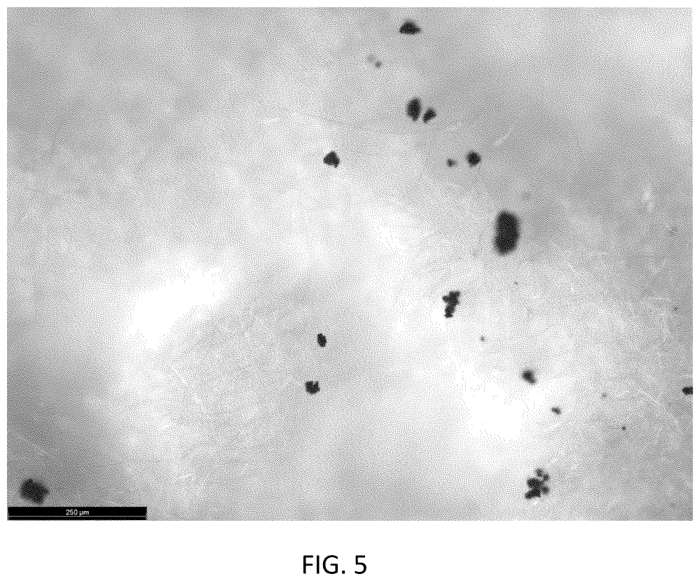

As shown in FIG. 3, a distinction was observed between tire wear and soot tested in the region of the thermogram, where the more combustion-resistant elemental carbon component was released during the PTA. Based on this result, soot and tire wear can be separated and distinguished based on the temperatures at which the elemental carbon fraction evolves in the thermogram. Tire wear samples were more thermally stable than soot under He/O₂ conditions, with oxidation not beginning until approximately 630° C. The temperature range over which the oxidation of tire wear occurred was broader (e.g., approximately 630° C. to 850° C. between 500 seconds to 629 seconds) when compared to that of soot. As shown in FIGS. 4A and 4B, soot appeared thermally “weak” relative to tire wear, and oxidation started occurring at approximately 550° C. to 630° C., between 449 seconds to 500 seconds under He/O₂ conditions. As shown in FIG. 5, the particle sizes of the cryo-milled tire wear examined ranged from 6 μm to 250 μm. Depending on the source, soot can have smaller particle sizes and higher surface areas, which can increase its ability to interact with other molecules (e.g., oxygen). Due at least in part to the larger number of carbon atoms exposed to oxygen molecules, the thermal stability of soot can decrease.

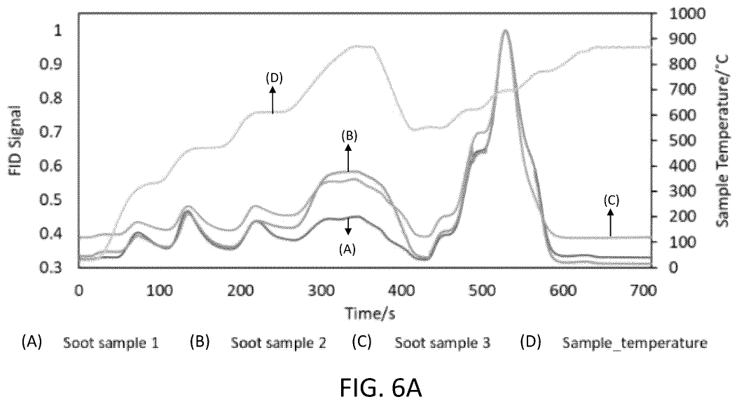

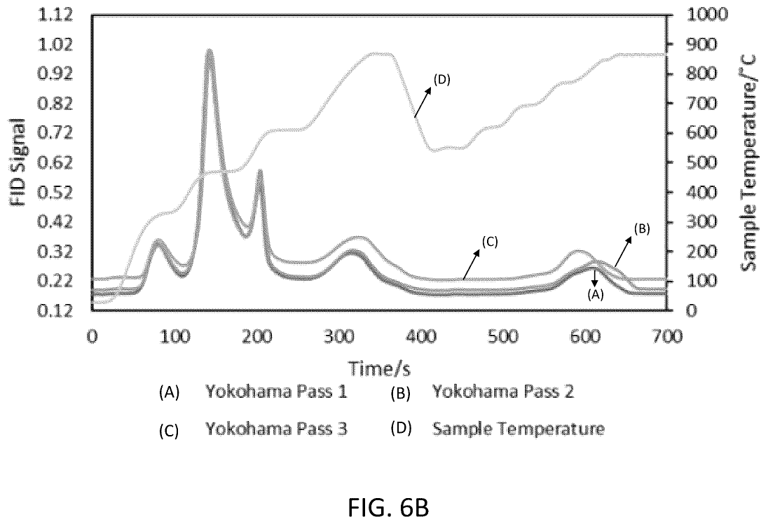

Repeatability measurements were made to assess consistency in the measurements. As shown in FIGS. 6A and 6B, soot and tire wear samples demonstrated repeatability when tested under the organic carbon-elemental carbon analyzer.

### Example 2. Effect of the Size of Tire Wear Particles on their Thermal Stability Under Oxidizing Carrier Gas Conditions

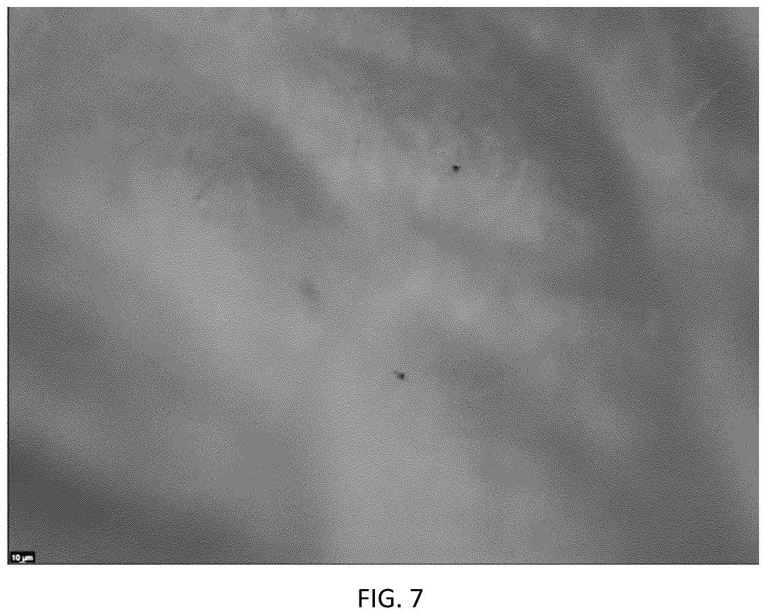

The temperatures at which the elemental carbon evolves in PTA was determined by the thermal stability of the sample. Smaller-sized particles can combust at lower temperatures due at least in part to their higher surface area/volume ratio, which allows them to efficiently transfer heat across their entire surface area. This facilitates the transport of oxygen molecules to the surface of particle which promotes faster combustion under oxidizing conditions. Therefore, the elemental carbon associated with the smaller particles is likely to be oxidized earlier in the thermal analyses. A tire wear sample subjected to longer cryo-milling grinding times with increased number of cycles produced particles ranging in size from 6 μm to 87 μm. As shown in FIG. 7, PM₁₀ filter samples from resuspension observed under the digital microscope showed particles with diameters less than 10 μm. Contrary to the expected results based on the aforementioned size effect on thermal stability of the sample, PM₁₀ of tire wear showed a separation from soot based on the temperatures at which the elemental carbon fraction evolves in the thermogram, as shown in FIGS. 4A and 4B. The compositions of soot and tire wear differ. For example, soot is more oxygenated compared to tire wear. Thus, compounds such as metal and sulfur impurities can adsorb to the surface of soot. In contrast, tire wear particles predominantly include rubber copolymers and additives, such as non-black fillers used to enhance tire performance and stability.

As shown in FIGS. 4A and 4C, carbon black N300 tire grade (30-35 nm) exhibited a distinction from soot under oxidizing carrier gas conditions in PTA. A similar observation was made for the recovered carbon blacks from waste tires (d<12 μm and d<26 μm) samples in the organic carbon-elemental carbon analyzer.

### Example 3. Application of the Tested Method on Urban Aerosol Samples

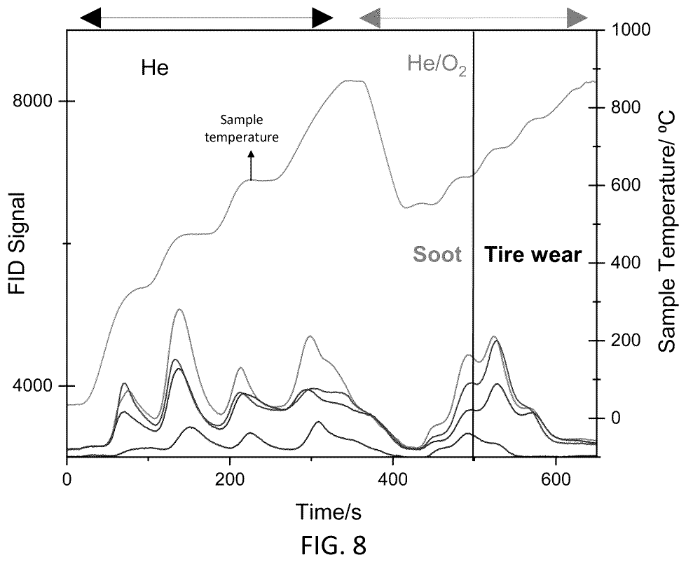

Ambient PM_{2.5} samples collected from the Tyler Street Parking structure showed the presence of a mixture of soot and tire wear, with elemental carbon evolving between approximately 550° C. to 800° C. in the thermograms, as shown in FIG. 8. Ambient aerosol samples collected in the vicinity of roads and urban areas can often contain a mixture of soot and tire wear particles. Tire wear generated as vehicles drive on roads can mix with the airborne soot generated from vehicle exhaust. The thermograms displayed high concentrations of soot and tire wear in the PM_{2.5} samples collected from the parking structure.

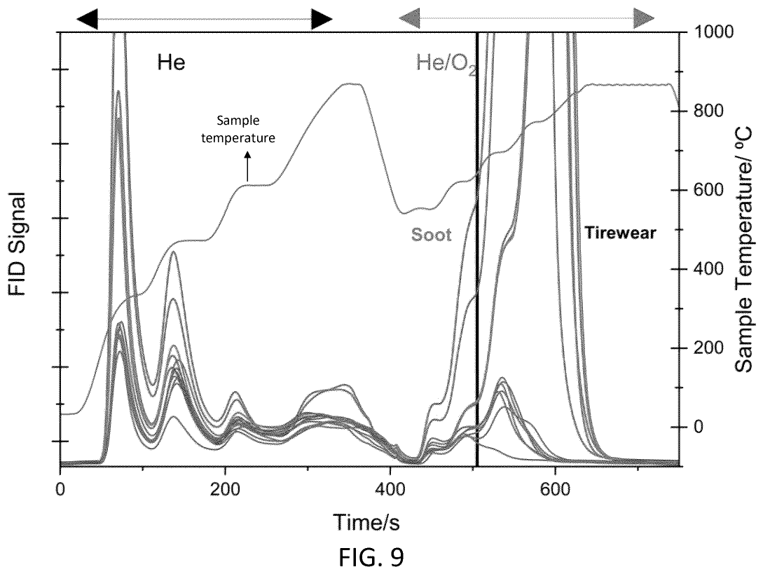

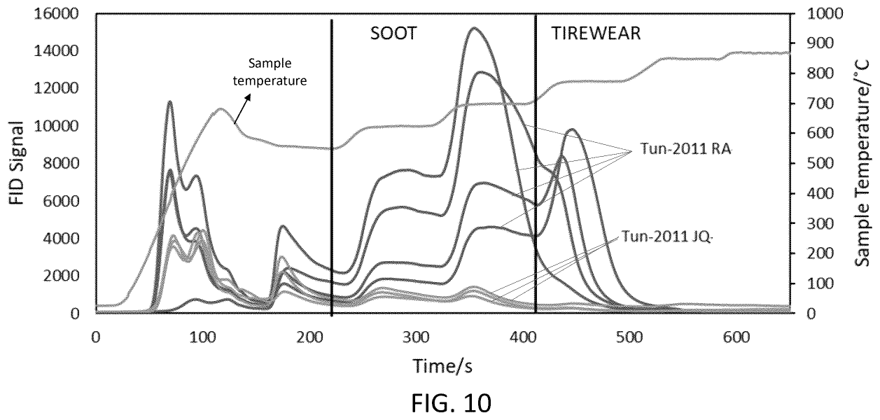

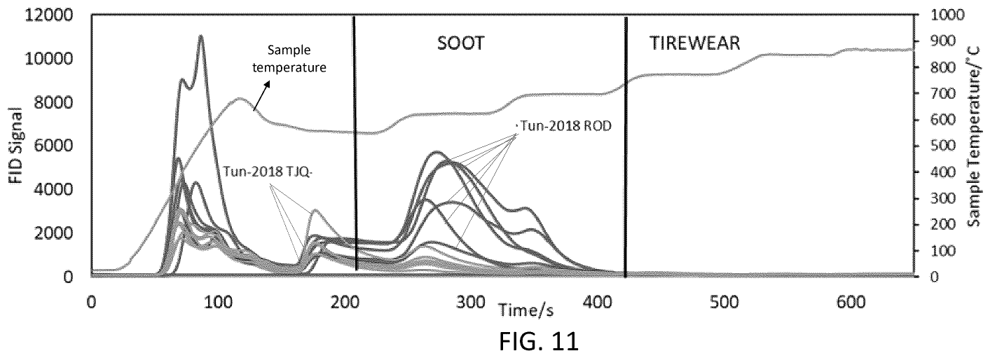

Referring to FIGS. 9, 10, and 11, a similar observation was made for the aerosol samples collected from two tunnels in the São Paulo Metropolitan Area, Brazil, which exhibited relatively higher elemental carbon content. A higher proportion of tire wear was observed compared to soot in the aerosol samples collected from both the tunnels. Lower soot and tire wear signatures were observed in ambient aerosol samples from the JQ tunnel, which witnesses mainly light duty vehicles, compared to those in the RA tunnel, where heavy duty vehicles were predominant.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method of identifying and quantifying tire wear, the method comprising:
heating a sample comprising particulate matter obtained from tire wear and soot to a first temperature to thermally desorb an amount of organic carbon from the sample;
cooling the sample from the first temperature to a second temperature;
heating the sample from the second temperature to a third temperature to thermally desorb an amount of elemental carbon from the sample; and
identifying an amount of tire wear particulate matter in the sample based on the amount of elemental carbon in the sample.

2. The method of claim 1, wherein the first temperature is in a range of about 600° C. to about 900° C.

3. The method of claim 1, wherein heating the sample to the first temperature occurs in the absence of oxygen.

4. The method of claim 1, wherein heating the sample to the first temperature comprises ramping a temperature of the sample from an initial temperature to the first temperature.

5. The method of claim 1, wherein the second temperature is in a range of about 500° C. to about 600° C.

6. The method of claim 1, wherein the third temperature is in a range of about 600° C. to about 900° C.

7. The method of claim 1, wherein heating the sample to the third temperature occurs in the presence of oxygen.

8. The method of claim 1, further comprising oxidizing the organic carbon to yield carbon dioxide.

9. The method of claim 8, further comprising converting the carbon dioxide to methane.

10. The method of claim 9, further comprising assessing an amount of the methane.

11. The method of claim 10, wherein assessing the amount of the methane comprises measuring the amount of methane using flame ionization detection.

12. The method of claim 11, wherein assessing the amount of the methane comprises integrating an area under a flame ionization detection curve between about 550° C. and about 630° C.

13. The method of claim 1, further comprising oxidizing the elemental carbon to yield carbon dioxide.

14. The method of claim 13, further comprising converting the carbon dioxide to methane.

15. The method of claim 14, further comprising assessing an amount of the methane.

16. The method of claim 15, wherein assessing the amount of the methane comprises measuring the amount of methane using flame ionization detection.

17. The method of claim 16, wherein assessing the amount of the methane comprises integrating an area under a flame ionization detection curve between about 630° C. and about 850° C.

18. The method of claim 15, further comprising adjusting the amount of methane to compensate for charring of the organic carbon in the sample.

19. The method of claim 1, wherein the method comprises programmed thermal analysis using an organic carbon-elemental carbon analyzer.
